(19) United States
(12) Patent Application Publication
(10) Pub. No.: US 2025/0264419 A1
(43) Pub. Date: Aug. 21, 2025

(54) RADIO-FREQUENCY SENSOR FOR INSTANTANEOUS WIRELESS BIOMARKER DETECTION

(71) Applicants: American University of Beirut, Beirut (LB); St Joseph University of Beirut, Beirut (LB)

(72) Inventors: Rayan Al Sayed Ali, Beirut (LB); Joseph Costantine, Albuquerque, NM (US); Youssef Tawk, Albuquerque, NM (US); Rouwaida Kanj, Cedar Park, TX (US); Assaad Eid, Paris (FR); Nassim Fares, Beirut (LB); Dania Chelala, Beirut (LB)

(21) Appl. No.: 19/052,625

(22) Filed: Feb. 13, 2025

Related U.S. Application Data
(60) Provisional application No. 63/554,560, filed on Feb. 16, 2024.

(51) Int. Cl. G01N 22/00; G01N 33/487; G01N 33/49; G01N 33/493; G01R 29/08; G01R 29/10

(52) U.S. Cl. CPC G01N 22/00; G01N 33/48778; G01N 33/49; G01N 33/493; G01R 29/0807; G01R 29/0878; G01R 29/10

(57) ABSTRACT

Provided herein are systems, apparatuses, and methods for a Radio-Frequency sensor for instantaneous wireless biomarker detection.

### BACKGROUND

The present application claims priority to U.S. Provisional Patent Application No. 63/554,560, filed Feb. 16, 2024, herein incorporated by reference in its entirety.

### BACKGROUND

The invention generally relates to a point-of-care device that can be used to measure various biomarkers in blood or urine. Hence, multiple applications are possible, including creatinine detection among others. There is currently a need for fast and accurate biomarker detection.

The present invention attempts to solve these problems, as well as others.

### SUMMARY OF THE INVENTION

Provided herein are systems, apparatuses, and methods for a Radio-Frequency sensor for

instantaneous wireless biomarker detection.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1A is a front perspective view of the sensor showing the two 2-turn exiting helices and the FR 4 base; FIG. 1B is a front perspective of the sensor showing the slotted conductive layer, the microstrip lines, and the ground planes two-port bifilar helix structure, exhibiting a cylindrical geometry. The bifilar helix has a modified inner structure to enhance sensing. FIG. 1C is a graph of the S11 magnitude of the sensor.

FIG. 2A is a front perspective view of the outer layer showing the two-port bifilar helix structure; FIG. 2B is a front perspective view of the sensor showing the second layer concentrically wrapped by the outer layer; FIG. 2C is a front perspective view of the third layer and the plurality slots within the inner metal cylinder; and FIG. 2D is a bottom perspective view of the supporting structure.

FIG. 3A is a Outer Layer: first layer composed of a bifilar helical structure including N=3 number of turns. The bifilar helical structure includes a width a=2 mm, a thickness=1 mm, a height h=33.7 mm, a pitch=9.9 mm; and FIG. 3B is a side profile view of the third layer and the plurality slots within the inner metal cylinder including N=1 of turns, a pitch=24 mm, a height h=33.7 mm, a thickness=1 mm, a separation distance between the outer slot b=4 mm and a width c=8 mm.

FIG. 4A is a front perspective view of the sensor showing the second layer and the fourth layer; FIG. 4B is an enlarged portion of FIG. 4A showing the base structure and the ground plane.

FIG. 5 is a photograph of the sensor 3D printed at PCB-way using additive manufacturing techniques.

FIG. 6A is Current density surface distribution of the sensor; and FIG. 6B is an Electric field distribution of the sensor.

FIGS. 7A-7C are photographs showing the testing of artificial plasma samples: (FIG. 7A) keysight FieldFox RF Vector Network Analyzer N9914A, (FIG. 7B) Empty sensor connected to VNA, (FIG. 7C) sensor loaded with artificial plasma samples.

FIGS. 8A-8D are graphs showing the S11 magnitude of the sensor for artificial plasma samples with distinct creatinine levels. FIG. 8E is a graph showing the predicted versus reference values for creatinine concentration for ten randomly shuffled test/train datasets used in Gaussian Process regression resulting in a Mean Absolute Relative Difference (MARD) of about 8.54%.

FIGS. 9A-9C are photographs of the diagram for testing of artificial urine samples: (FIG. 9A) keysight FieldFox RF Vector Network Analyzer N9914A, (FIG. 9B) Empty sensor connected to VNA, (FIG. 9C) sensor loaded with artificial urine samples.

FIGS. 10A-10B are graphs showing the S11 and S21 magnitude of the sensor for artificial urine samples with distinct creatinine levels.

FIGS. 11A-11B are graphs showing the sensor's response to creatinine variation during artificial urine testing. The S11 magnitude versus the reference creatinine levels at the distinct frequencies: (FIG. 11A) about 4 GHz and (FIG. 11B) about 4.225 GHz. The green line is the S11 fitted curve showing the trend of the sensor's response when the creatinine concentrations increase.

FIGS. 12 is a graph showing the predicted creatinine concentration in artificial urine versus reference creatinine level with a MARD of about 11.96%.

FIG. 13A is a graph showing the predicted creatinine concentration versus reference creatinine level for urine samples collected from rats at baseline, FIG. 13B is a graph showing the predicted creatinine concentration versus reference creatinine level for urine samples collected from rats 1 month after injection, and FIG. 13C is a graph showing the predicted creatinine concentration versus reference creatinine level for urine samples collected from rats 2 months after injection with an about 6.94%, about 10%, and about 7.28% MARD of prediction, respectively.

FIG. 14 is a graph showing the predicted creatinine concentration in human urine versus reference creatinine level with a MARD of about 7.3%.

FIG. 15 is a graph showing the predicted creatinine concentration in human plasma versus reference creatinine level with a MARD of about 3.28%.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, electrical, biological, biochemical, mechanical, and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Description of the Embodiments

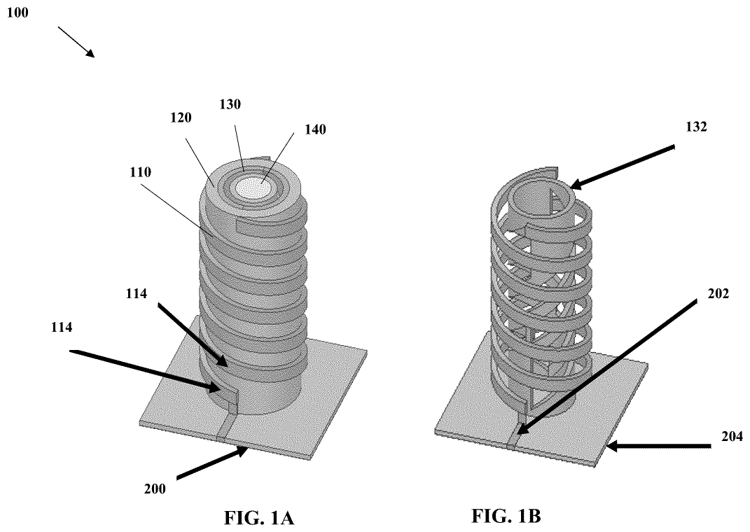

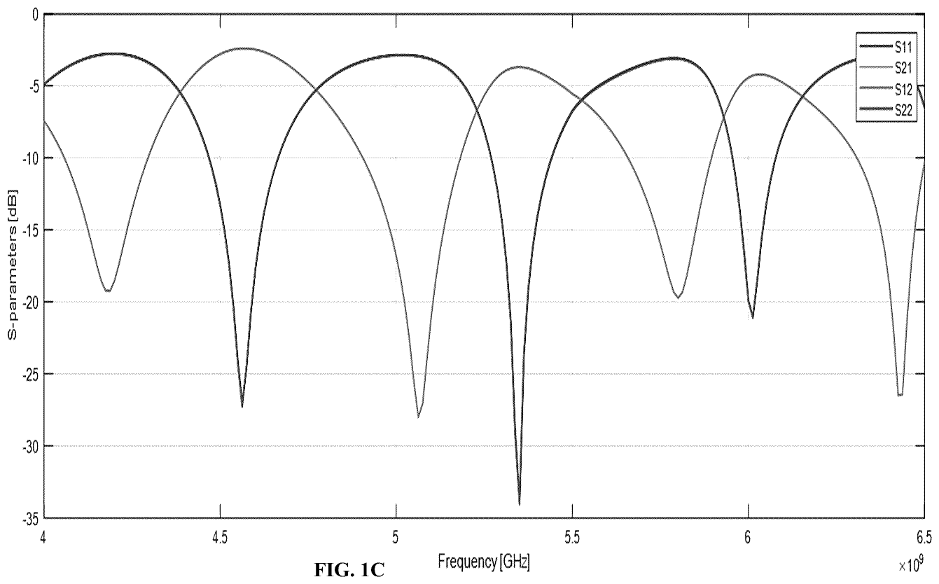

Generally speaking, a radio-frequency sensor for instantaneous wireless biomarker detection sensor 100 comprises a two-port bifilar helix structure, exhibiting a cylindrical geometry, as shown in FIG. 1A. The bifilar helix has a modified inner structure to enhance sensing. In one embodiment, the sensor is a 3D structure that has volumetric sensitivity towards change in the composition of urine and blood rather than planar sensitivity.

A method for instantaneous wireless detection of biomarkers, comprises: using a two-port bifilar helix antenna-based structure, and including a modified inner structure to enhance sensing with the two-port bifilar helix structure; detecting the composition of urine or blood by using volumetric sensitivity towards a change in the composition of urine or blood rather than planar sensitivity. The method further comprises concentrically wrapping four cylindrical layers around each other; including an outer layer in the two-port bifilar helical structure, wrapping the outer layer around a second layer; including a plastic cylindrical column and a volumetric structure in the second layer; placing a third layer inside the plastic cylindrical column and the third layer composed of a conductive metal inner cylinder as the sensing layer of the sensor, and including a plurality of slots in the conductive metal inner cylinder; providing a fourth layer within the third layer, and including a plastic tube within the sensing layer, and holding the sample in the plastic tube during measurements.

The method further comprises the two-port bifilar helix structure as an external feeder helix with two 3-turn helices and radiates azimuthal direction. The method further comprises orienting the plurality of slots in the conductive metal inner cylinder as orthogonal to the rotation orientation of the two-port bifilar helix structure to enhance sensing, and the radiation from two-port bifilar helix structure including the reflected fields from the conductive metal inner cylinder leak through the plurality of slots to amplify the sensing capability and to focus the signal on the inner testing of the fourth layer.

The method further comprises supporting the cylindrical section by a base structure that is composed of 2D layers including a feeding network, a dielectric substrate, and a ground plane. The method further comprises using the cylinder slots as complementary split resonators and detecting the concentration of urine constituents instantaneously through electromagnetic wave radiation; and urine constituents include creatinine, ammonia, urea, or uric acid; and detecting creatinine between about 0.2 mg/dl and about 4 mg/dl.

The method further comprises monitoring the creatinine level over multiple frequency bands with enhanced sensitivity with the plurality of slots in the conductive metal inner cylinder leak result in a multi-band response. The method further comprises providing a current density surface distribution along with the electric field distribution over the inner surface of the slotted metallic cylinder that surrounds the sample shows highly concentrated areas around the slots of the inner metallic cylinder of the sensor at different frequencies, including a highly sensitive area towards changes in the constituents of the loading sample.

As shown in FIGS. 1A-1B, the sensor 100 comprises a volumetric cylindrical tube including 4 cylindrical layers wrapped around each other and disposed on a base 200, wherein the outer layer 110 is concentrically wrapped around a second layer 120, the second layer 120 is concentrically wrapped around a third layer 130, and the third layer 130 is concentrically wrapped around a fourth layer. The outer layer 110 is composed of a bifilar helical structure 112 including two 3-turn exciting helices 114. The bifilar helical structure 112 is wrapped around a second layer 120 composed of a PLA plastic cylindrical column, as shown in FIG. 3B. The conductive metal inner cylinder 120 is the sensing layer of the sensor system and comprises a plurality of slots 134, as shown in FIG. 3C. The third layer 130 is composed of a conductive metal inner cylinder 132, which is inside the PLA plastic cylindrical column. The fourth layer 140 is composed of a PLA plastic tube within the conductive metal inner cylinder 132. The PLA plastic tube placed within the sensing volume of the sensor, and responsible for holding the sample during measurements.

FIG. 1B is a front perspective of the sensor showing the slotted conductive layer, the microstrip lines, and the ground planes two-port bifilar helix structure, exhibiting a cylindrical geometry. The bifilar helix has a modified inner structure to enhance sensing. FIG. 1C is a graph of the S11 magnitude of the sensor.

As shown in FIG. 3A, the external bifilar helix structure 112 is an external feeder helix that is composed of two 3-turn helices 114 and radiates in the azymuthal direction. The external bifilar helix structure 112 terminates on the base 200 with a feeding line 116. As shown in FIG. 3C, the third cylinder 130 includes the plurality of slots 134 that are orthogonal to the rotation orientation of the external helix structure 112 to enhance sensing. Radiation from feeder helix including the reflected fields from the slotted metallic layer 130 will leak through the slots which amplifies the sensing capability. This allows to focus the signal on the inner testing apparatus.

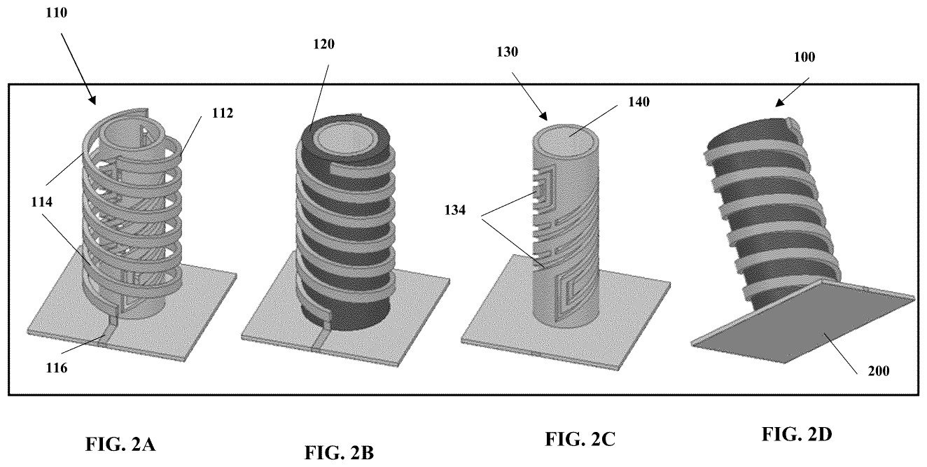

As shown in FIG. 2D, the cylindrical sensor 100 is supported by a base structure 200 that is composed of 2D layers including the feeding network 116 (two micro-strip lines 202 feeding the helices), a dielectric substrate (FR4), and a ground plane 204.

The volumetric cylindrical tube is composed of 4 cylindrical layers coaxially wrapped around each other. In one embodiment, the outer layer includes dimensions with a radius=about 8 mm, a height=about 33.7 mm, and a thickness=about 1 mm. The second layer includes dimensions with a radius=about 6.01 mm, a height=about 33.7 mm, and a thickness=about 2 mm. The third layer includes dimensions with a radius=about 5.01 mm, a height=about 33.7 mm, and a thickness=about 1 mm. The fourth layer includes dimensions with a radius=about 3.01 mm, a height=about 33.7 mm, and a thickness=about 2 mm. Alternative dimensions may be included as long as the cylindrical layers are coaxially wrapped around each other.

As shown in FIG. 3B, the cylinder slots are complementary split resonators, to detect the concentration of urine constituents instantaneously through electromagnetic wave radiation. Urine constituents include, and are not restricted to creatinine, ammonia, urea, and uric acid.

FIG. 3A is first layer composed of a bifilar helical structure including N=3 number of turns. The bifilar helical structure includes a width a=about 2 mm, a thickness=about 1 mm, a height h=about 33.7 mm, a pitch=about 9.9 mm; and FIG. 3B is a side profile view of the third layer and the plurality slots within the inner metal cylinder including N=1 of turns, a pitch=about 24 mm, a height h=about 33.7 mm, a thickness=about 1 mm, a separation distance between the outer slot b=about 4 mm and a width c=about 8 mm. Alternative dimensions may be included as long as the N number of turn remains consistent.

In one embodiment, the sensor design operates in the lower microwave frequency band between about 4 GHz and about 6.5 GHz.

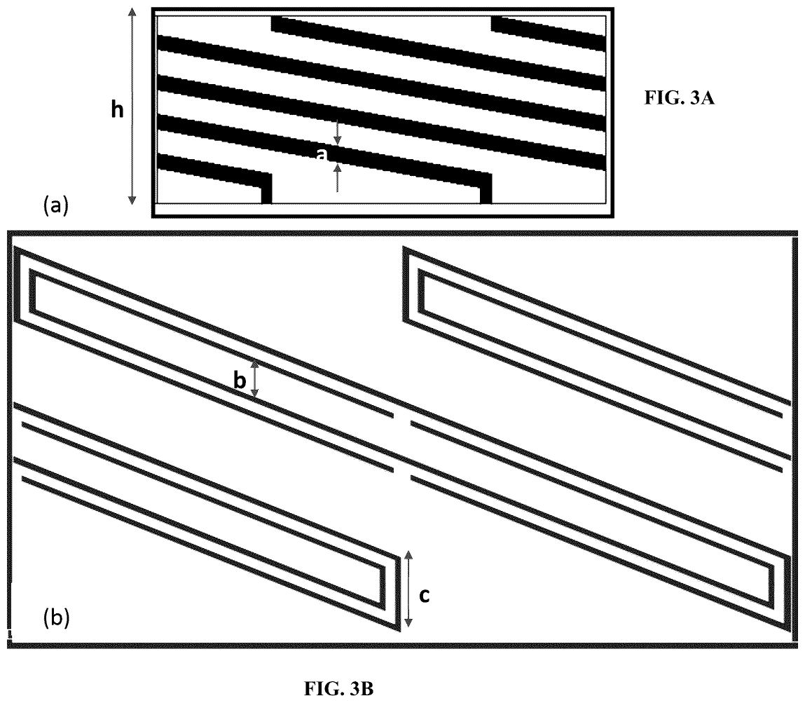

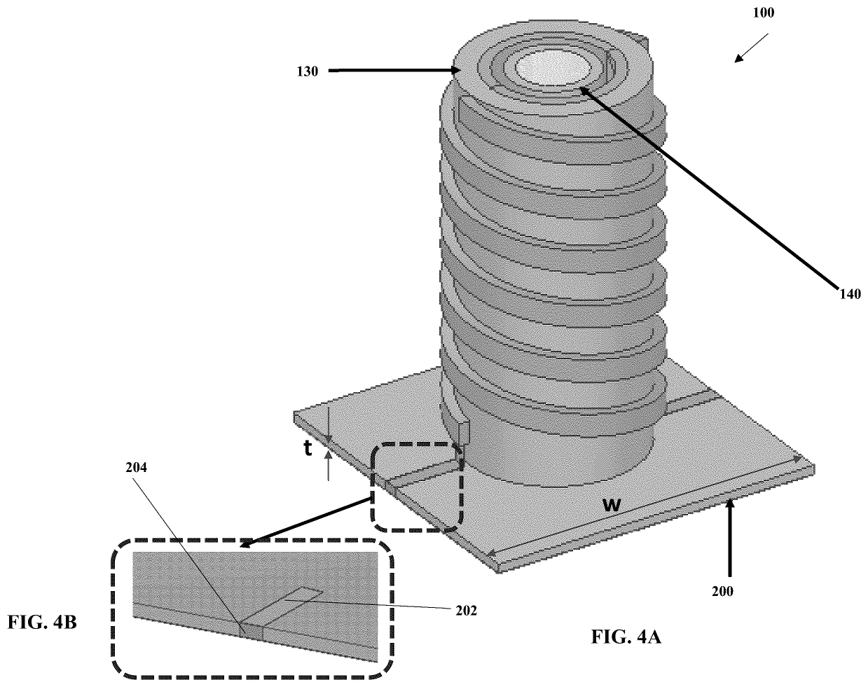

As shown in FIG. 4A-4B, the second layer 120 acts as the substrate separating the two conductive layers 1 and 3. The second layer is made of PLA plastic and includes the dimensions of height h=about 33.7 mm and a thickness=about 2 mm. The fourth layer 140 acts as a sample holder and is made of PLA plastic. The fourth layer 140 includes dimensions of height h=about 33.7 mm and a thickness=about 2 mm. The base structure 200 includes 2D layers including the feeding network, a dielectric substrate (FR4) 202, and a ground plane 204. The base structure 200 includes a width w=about 30 mm, and a thickness t=about 0.8 mm. Alternative dimensions may be included as long as the cylindrical layers are coaxially wrapped around each other.

The sensing tube is surrounded by multiple slots, resulting in a multi-band response, which allows the monitoring of the creatinine level over multiple frequency bands with enhanced sensitivity.

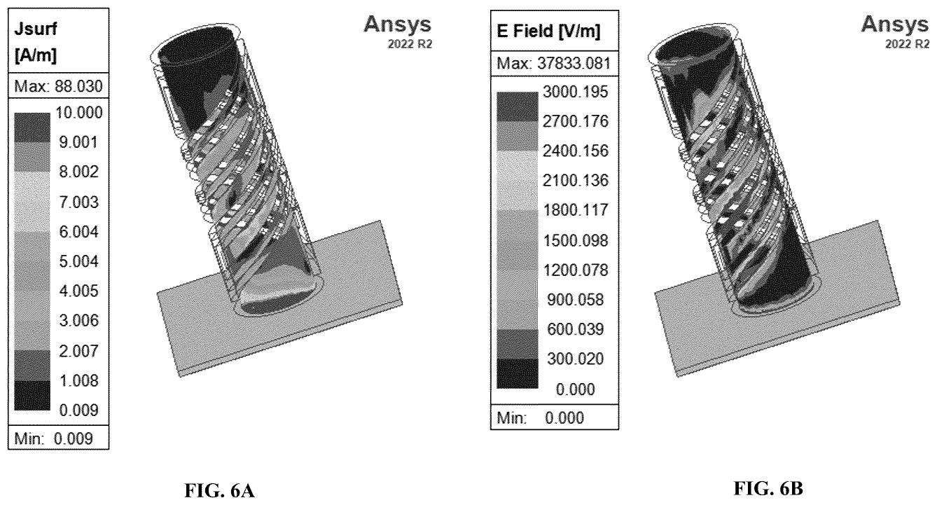

As shown in FIG. 6A-6B, the current density surface (Jsurf) distribution along with the electric field distribution (E field) over the inner surface of the slotted metallic cylinder that surrounds the sample show highly concentrated areas around the slots of the inner metallic cylinder of the sensor at different frequencies, which implies highly sensitive area towards changes in the constituents of the loading sample. The E field includes a density of about 3000.195 V/m and the current density surface Jsurf of 10 A/m over the inner surface of the slotted metallic cylinder. Surface current density of the antenna

The two signals measured from the sensor, upon loading a urine sample under test into it, are analyzed using a computer program, that utilizes regression modeling techniques, to map the recorded scattering parameters (magnitude and phase) to concentration of the urine sample constituents.

The sensor system is designed to present a high correlation between the S-parameters response recorded upon loading the sensor with a urine or blood sample, and the variation in the concentration of the constituents of the loading sample.

The sensor is tested in pre-clinical and clinical settings and has reported high sensitivity toward changes in creatinine concentration of a urine sample or blood sample. The conducted experiments support and enable the sensor's capability to accurately detect the creatinine concentration of the loading sample.

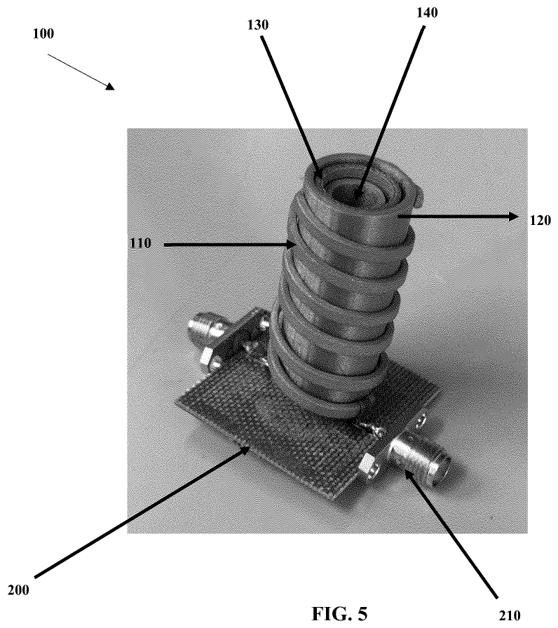

As shown in FIG. 5, the outer layer 110 and the third layer 130 are 3D printed at PCB-way using additive manufacturing techniques. Aluminum (AlSi 10 Mg) was used along with Selective laser melting (SLM) technique to execute the 3D printing. The second layer 120 and the third layer 130 are 3D printed using PLA plastic material. The base structure 200 is fabricated on a FR4 epoxy substrate using LPKF S63 CNC milling machine.

Other biomarkers that could detected include biological markers that are novel/foreign/malignant or non-malignant cells or other newly developed molecules that may not be part of the typical constituents of the biological system. Biomarkers can also be traced not only in blood, but in the rest of the biological system, such as saliva, tissue, and the like.

Biomarkers as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Biomarkers can include naturally occurring substances, artificial substances, metabolites, and/or reaction products. In some embodiments, the biomarker for measurement by the sensor, devices, and methods is a biomarker. However, other biomarkers are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, Leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute biomarkers in certain embodiments. The biomarkers can be naturally present in the biological fluid, for example, a metabolic product, a hormone, an antigen, an antibody, and the like. Alternatively, the biomarker can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex,

Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated biomarkers. Biomarkers such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Regression Modeling

The sensor is connected to a vector network analyzer (VNA) to record its magnitude and/or the phase response towards the change in the concentration of biomarker constituents of a loading blood or urine sample. The experiment procedure is conducted as follows:

Data collection, i.e., Measurements of the S-parameters response of the sensor. The acquired data consists of the magnitude and phase of S11, S21 and S22 which correspond to the distinct frequencies obtained from the VNA over the range from about 4 to about 6.5 GHZ, as shown in FIG. 1C.

Data Preprocessing: Each VNA measurement was comprised as the average of 10 readings to avoid the VNA's inherent random noise.

Sampling: To minimize redundancy between recorded features, the frequency range was sampled with an about 0.0625 GHz frequency step within the operating frequency range from about 4 GHz to about 6.5 GHz.

Normalization: For each Feature, the value of the S-parameter magnitude or phase was normalized to a value between 0 and 1.

The resulting data are then used to train a Gaussian process (GP) regression model that maps the device parameters to estimate the concentration of the desired biomarker.

The model relies on S-parameters values obtained at critical frequencies as identified by a wrapper feature selection technique. In one embodiment, forward feature selection (FFS) wrapper method is used, through which an optimal feature subset is selected based on the regression model output. The initial optimal feature subset has zero features, and for each iteration, the feature that pro-vides the lowest model output mean error is added to the subset. Although this feature selection method is computationally expensive, it can provide an excellent selection of the sensitive features.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Exampled 1: Experiments on Artificial Plasma Samples

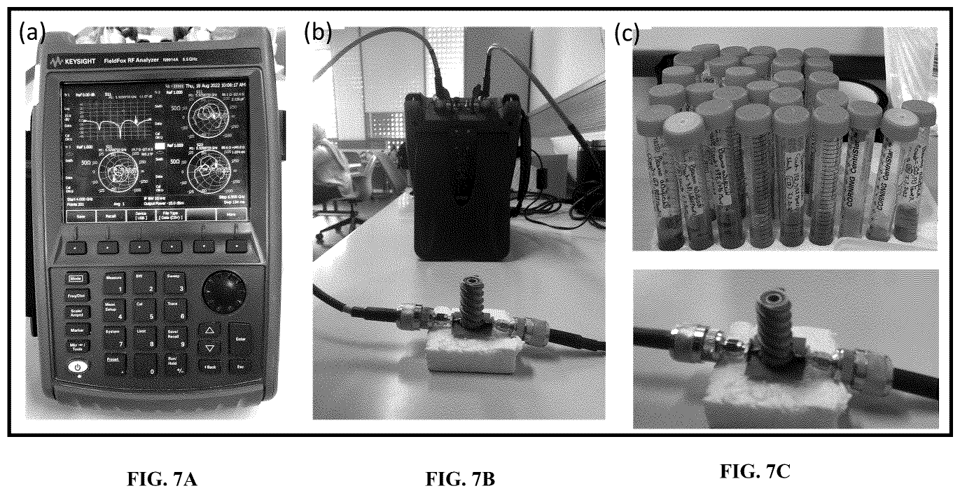

FIGS. 7A-7C is a diagram showing testing of artificial plasma samples: (FIG. 7A) keysight FieldFox RF Vector Network Analyzer N9914A, (FIG. 7B) Empty sensor connected to VNA, (FIG. 7C) sensor loaded with artificial plasma samples.

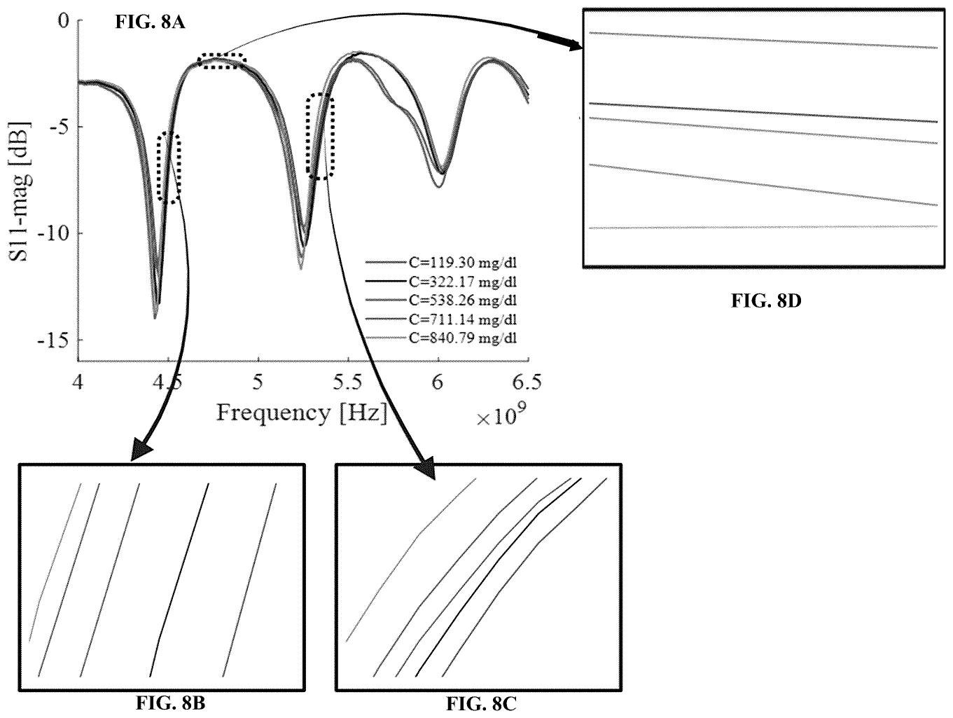

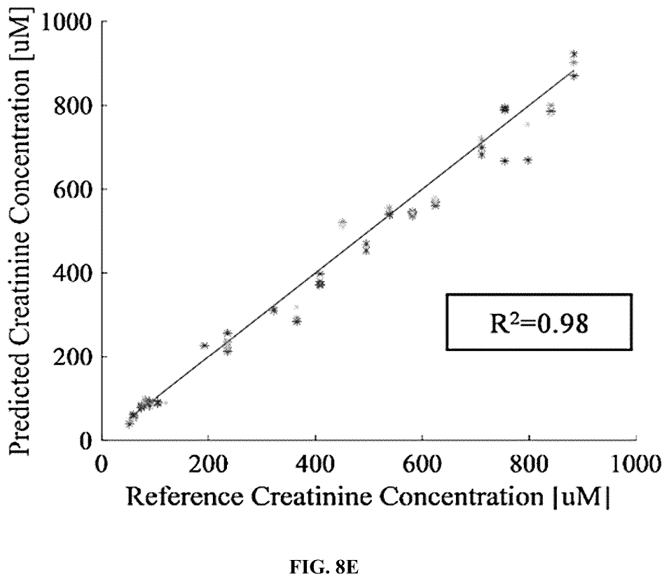

FIGS. 8A-8D is a graph showing S11 magnitude of the sensor for artificial plasma samples with distinct creatinine levels and frequencies between 4 Hz and 6.5 Hz. The S11 magnitude at 4.5 Hz is between about −8 [dB] and about −5 [dB] as shown in FIG. 8B. The S11 magnitude at 5.5 Hz is between about −7 [dB] and about −4 [dB] as shown in FIG. 8C. The S11 magnitude at 5.0 Hz is between about −3 [dB] and about −2 [dB] as shown in FIG. 8D. FIG. 8E is a graph showing the predicted versus reference values for creatinine concentration for ten randomly shuffled test/train datasets used in Gaussian Process regression resulting in a mean data error or Mean absolute relative difference (MARD) of about 8.54% with an R²=about 0.98. The embodiment of the present sensor detected creatinine levels of 119.30 mg/dl, 322.17 mg/dl, 538.26 mg/dl, 711.14 mg/dl, and 840.79 mg/dl. Mean absolute relative difference a statistical metric that compares the accuracy of a sensor or other device to a reference measurement. MARD is often used to evaluate the accuracy. MARD is calculated by averaging the absolute differences between the sensor measurement and the reference measurement. MARD is reported as a percentage. A lower MARD indicates better agreement between the sensor and the reference measurement.

### Example 2: Experiments on Artificial Urine Samples

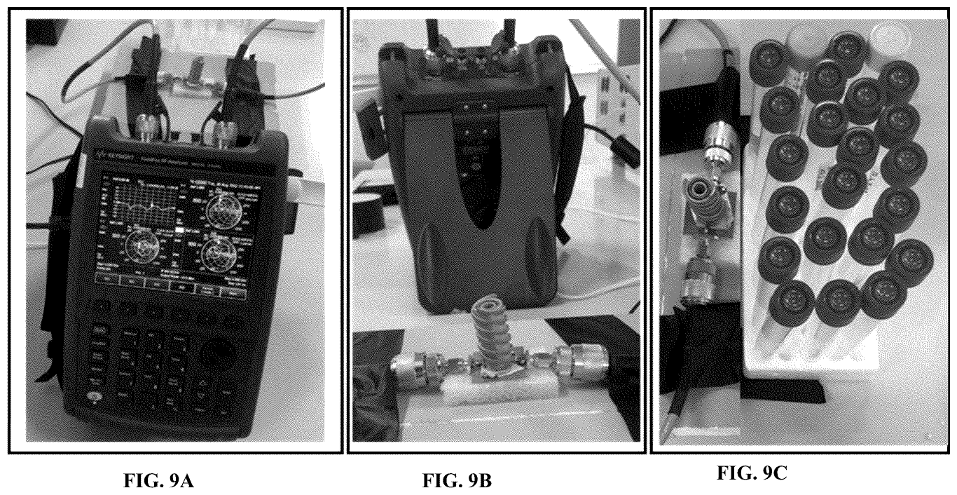

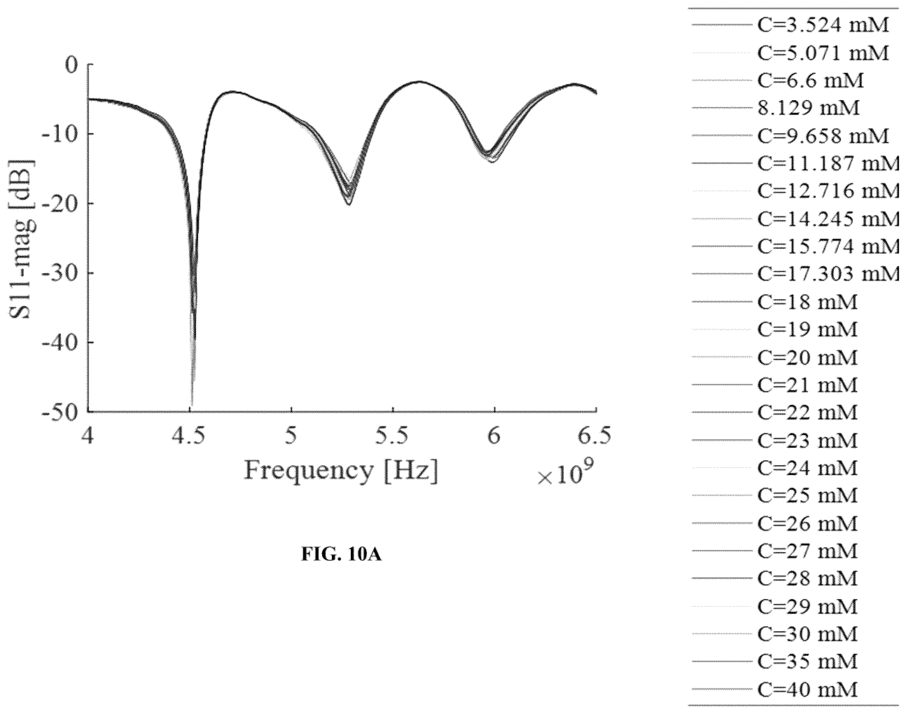

FIGS. 9A-9C show the testing setup of artificial urine samples: (FIG. 9A) keysight FieldFox RF Vector Network Analyzer N9914A, (FIG. 9A) Empty sensor connected to VNA, (FIG. 9C) sensor loaded with artificial urine samples.

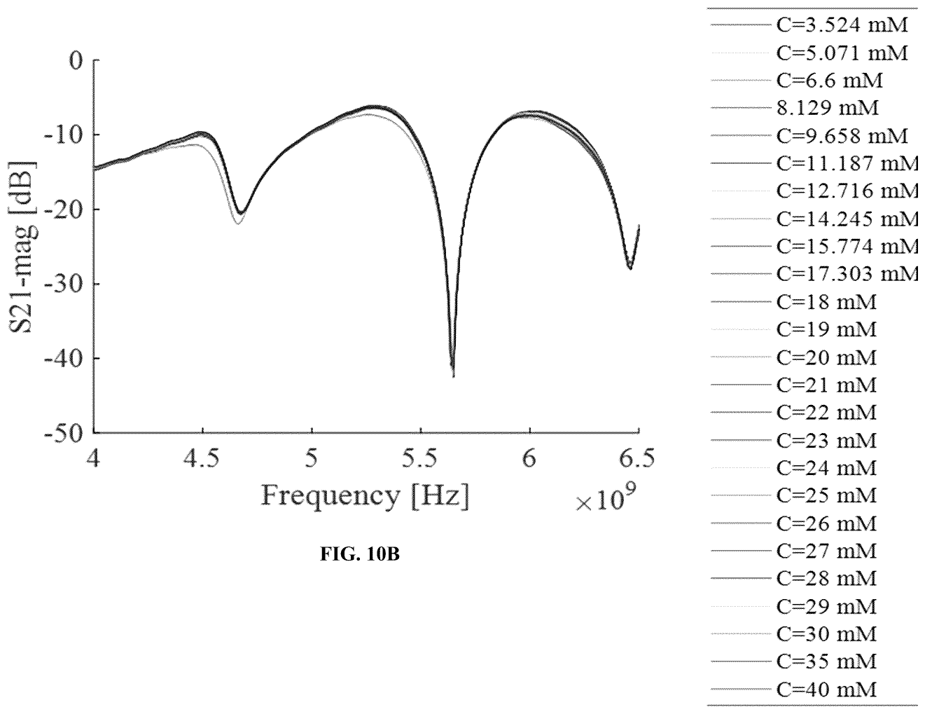

FIGS. 10A-10B are graphs showing the S11 and S21 magnitude of the sensor for artificial urine samples with distinct creatinine levels. At frequencies between about 4 and about 6.5 Hz and S11 magnitude between −3 [dB] and −50 [dB], the sensor of the present invention detected creatinine levels at 3.524 mM, 5.071 mM, 6.6 mM, 8.129 mM, 9.658 mM, 11.187 mM, 12.716 mM, 14.245 mM, 15.774 mM, 17.303 mM, 18, mM, 19 mM, 20 mM, 21 mM, 22 mM, 23 mM, 24 mM, 25 mM, 26 mM, 27 mM, 28 mM, 29 mM, 30 mM, 35 mM, and 40 mM, as shown in FIG. 10A. At frequencies between 4 and 6.5 Hz and S21 magnitude between −5 [dB] and −50 [dB], the sensor of the present invention detected creatinine levels at 3.524 mM, 5.071 mM, 6.6 mM, 8.129 mM, 9.658 mM, 11.187 mM, 12.716 mM, 14.245 mM, 15.774 mM, 17.303 mM, 18, mM, 19 mM, 20 mM, 21 mM, 22 mM, 23 mM, 24 mM, 25 mM, 26 mM, 27 mM, 28 mM, 29 mM, 30 mM, 35 mM, and 40 mM, as shown in FIG. 10B.

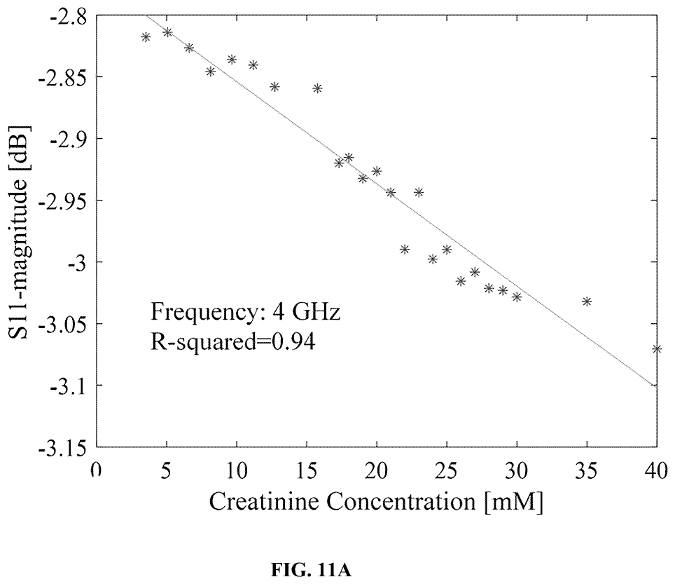

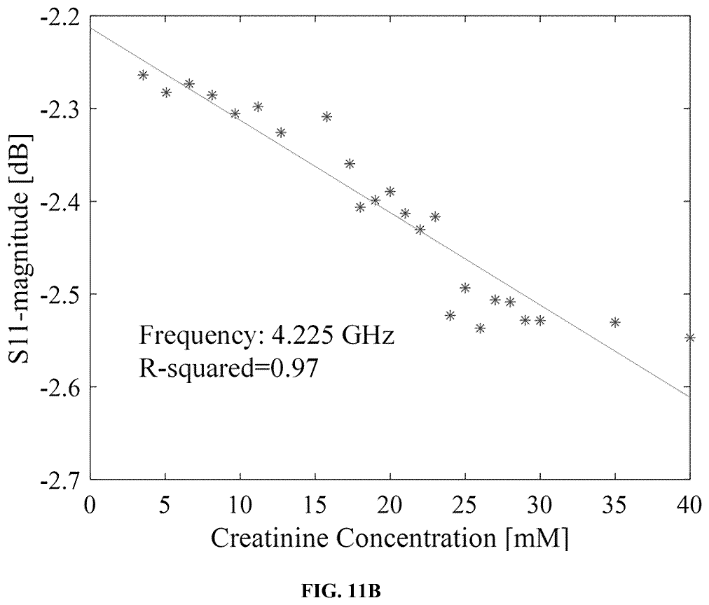

FIGS. 11A-11B are graphs showing the sensor's response to creatinine variation during artificial urine testing. The S11 magnitude between −2.8 [dB] and −3.1 [dB] versus the reference creatinine levels at the distinct frequencies: (FIG. 11A) 4 GHz with an R² at 0.94 for creatinine concentrations between 5 and 40 mM; and (FIG. 11B) 4.225 GHz with an R² at 0.97 for creatinine concentrations between 5 and 40 mM. The green line is the S11 fitted curve showing the trend of the sensor's response when the creatinine concentrations increase.

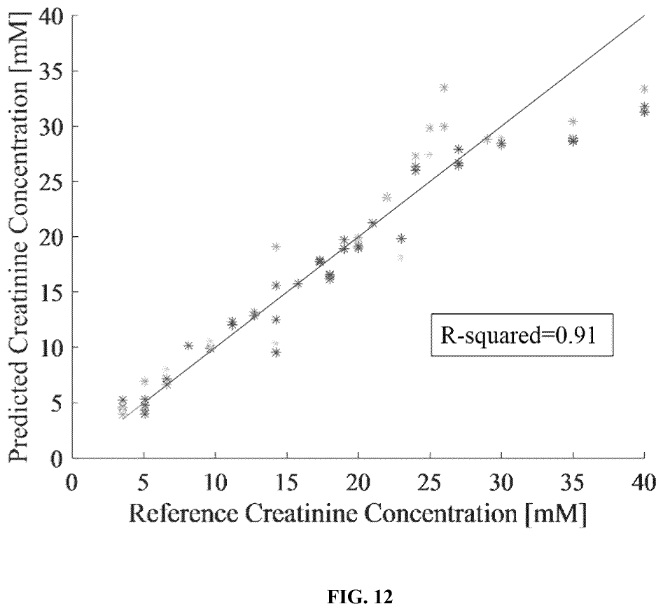

FIG. 12 is a graph showing the predicted creatinine concentration between 5 and 40 mM in artificial urine versus reference creatinine level between 5 and 40 mM with a MARD of 11.96% with an R² at 0.91. This indicates a high accuracy at detecting creatinine concentrations between 5 and 40 mM in urine.

### Example 3: In-Vivo Experiments on Animal Models

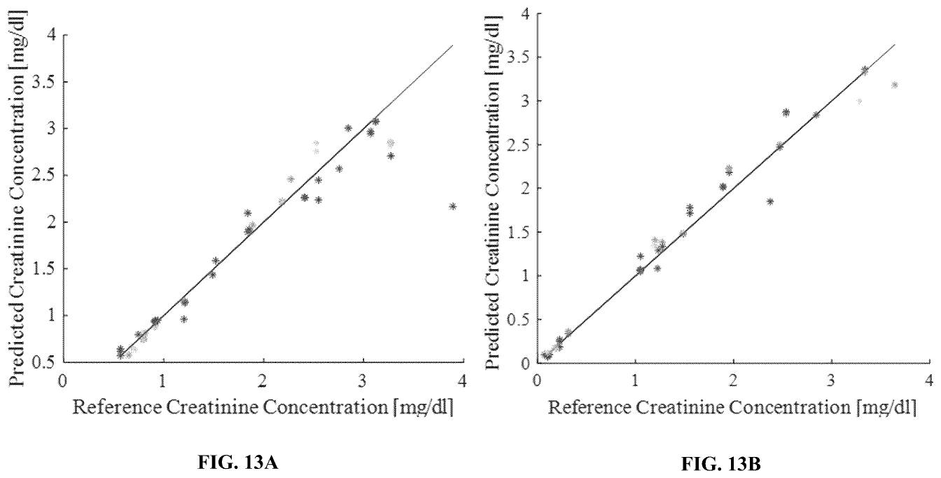

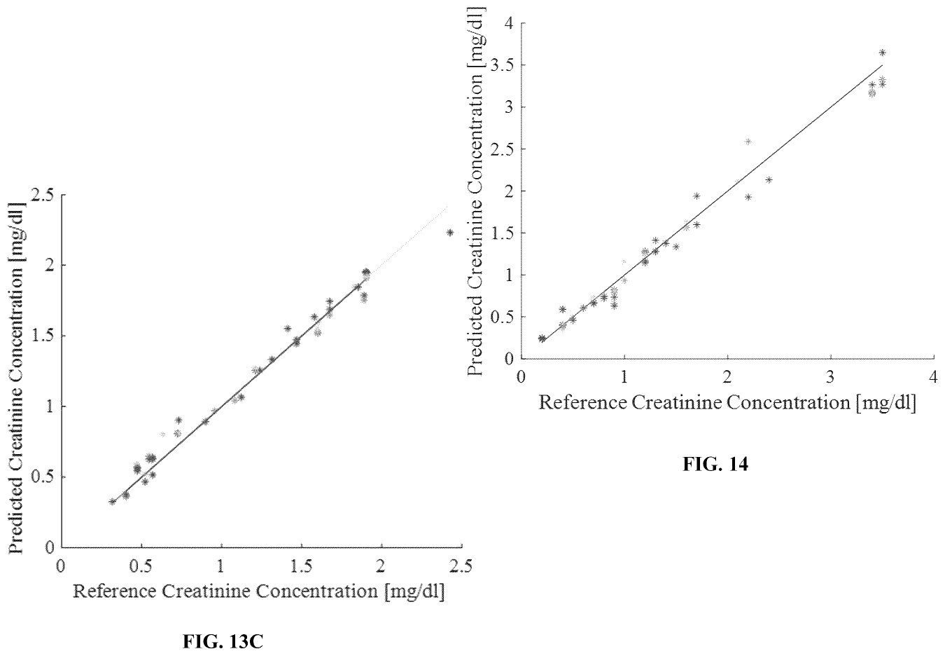

FIG. 13A-13C are graphs showing (FIG. 13A) Predicted creatinine concentration between about 0.5 mg/dl and about 4 mg/dl and versus reference creatinine levels between 0 and 4 mg/dl for urine samples collected from rats at baseline, (FIG. 13B) Predicted creatinine concentration about 0.5 mg/dl and about 4 mg/dl versus reference creatinine levels between 0 and 4 mg/dl for urine samples collected from rats 1 month after injection, (FIG. 13C) Predicted creatinine concentration about 0.5 mg/dl and about 2.5 mg/dl versus reference creatinine levels between 0 and 2.5 mg/dl for urine samples collected from rats 2 months after injection, with an about 6.94%, about 10%, and about 7.28% MARD of prediction, respectively.

### Example 4: Clinical Studies—Human Urine

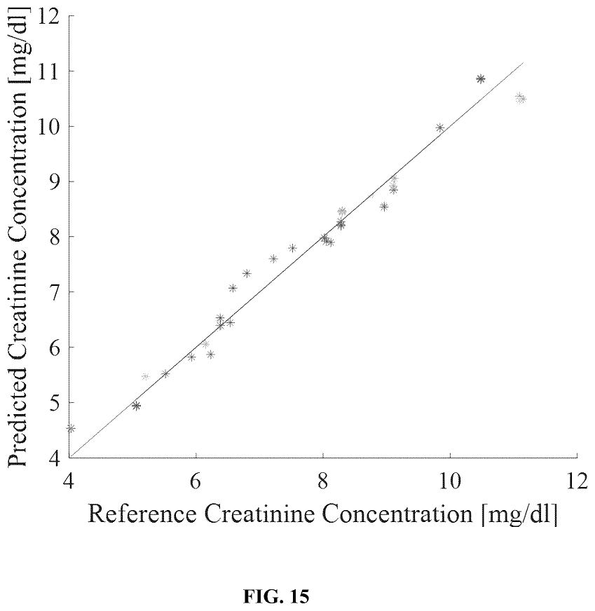

FIG. 14 is a graph of the predicted creatinine concentration between about 0.2 mg/dl and about 4 mg/dl in human urine versus reference creatinine levels between 0.2 and 4 mg/dl with a MARD of about 7.3%.

### Exampled 5: Clinical Studies—Human Serum

FIG. 15 is a graph of the predicted creatinine concentration between about 4 mg/dl and about 10 mg/dl in human plasma versus reference creatinine levels between about 4 mg/dl and about 12 mg/dl with a MARD of about 3.28%.

### System

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

In some embodiments, some or all of the functions or process(es) described herein and performed by a computer program that is formed from computer readable program code and that is embodied in a computer readable medium. The phrase “computer readable program code” includes any type of computer code, including source code, object code, executable code, firmware, software, etc. The phrase “computer readable medium” includes any type of medium capable of being accessed by a computer, such as read only memory (ROM), random access memory (RAM), a hard disk drive, a compact disc (CD), a digital video disc (DVD), or any other type of memory.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A sensor comprising: a two-port bifilar helix antenna-based structure, the two-port bifilar helix structure includes a modified inner structure to enhance sensing; wherein the sensor is a 3D structure that has volumetric sensitivity towards change in the composition of urine and blood rather than planar sensitivity.

2. The sensor in claim 1, further comprises four cylindrical layers concentrically wrapped around each other; an outer layer including the two-port bifilar helical structure, the outer layer wrapped around a second layer; the second layer including a plastic cylindrical column and a volumetric structure; a third layer inside the plastic cylindrical column and the third layer composed of a conductive metal inner cylinder as the sensing layer of the sensor, and the conductive metal inner cylinder comprises a plurality of slots; a fourth layer within the third layer, the fourth layer includes a plastic tube placed within the sensing layer of the sensor, and responsible for holding the sample during measurements.

3. The sensor of claim 2, wherein the two-port bifilar helix structure is an external feeder helix that is composed of two 3-turn helices and radiates azimuthal direction.

4. The sensor of claim 3, wherein the plurality of slots in the conductive metal inner cylinder are orthogonal to the rotation orientation of the two-port bifilar helix structure to enhance sensing, and radiation from two-port bifilar helix structure including the reflected fields from the conductive metal inner cylinder leak through the plurality of slots to amplify the sensing capability and to focus the signal on the inner testing of the fourth layer.

5. The sensor of claim 4, wherein the cylindrical section is supported by a base structure that is composed of 2D layers including a feeding network, a dielectric substrate, and a ground plane.

6. The sensor of claim 5, wherein the cylinder slots are complementary split resonators, to detect the concentration of urine constituents instantaneously through electromagnetic wave radiation; and urine constituents include creatinine, ammonia, urea, or uric acid.

7. The sensor of claim 6, wherein the sensor operates in the lower microwave frequency band between about 4 GHz and about 6.5 GHz.

8. The sensor of claim 7, wherein the plurality of slots in the conductive metal inner cylinder leak result in a multi-band response, which monitors the creatinine level over multiple frequency bands with enhanced sensitivity.

9. The sensor of claim 8, further comprising a current density surface distribution along with the electric field distribution over the inner surface of the slotted metallic cylinder that surrounds the sample shows highly concentrated areas around the slots of the inner metallic cylinder of the sensor at different frequencies, including a highly sensitive area towards changes in the constituents of the loading sample.

10. The sensor of claim 9, further comprising two signals measured from the sensor, upon loading a urine sample under test into it, are analyzed using a computer program, that utilizes regression modeling techniques, to map the recorded scattering magnitude and phase parameters to concentration of the urine sample constituents.

11. The sensor of claim 10, further comprises a high correlation between the signals measured from the sensor (S-parameters) response recorded upon loading the sensor with a urine or blood sample, and the variation in the concentration of the constituents of the loading sample.

12. The sensor of claim 10, further comprising a high sensitivity toward changes in creatinine concentration of a urine or blood sample.

13. A method for instantaneous wireless detection of biomarkers, comprising: using a two-port bifilar helix antenna-based structure, and including a modified inner structure to enhance sensing with the two-port bifilar helix structure; detecting the composition of urine or blood by using volumetric sensitivity towards a change in the composition of urine or blood rather than planar sensitivity.

14. The method of claim 13, further comprises concentrically wrapping four cylindrical layers around each other; including an outer layer in the two-port bifilar helical structure, wrapping the outer layer around a second layer; including a plastic cylindrical column and a volumetric structure in the second layer; placing a third layer inside the plastic cylindrical column and the third layer composed of a conductive metal inner cylinder as the sensing layer of the sensor, and including a plurality of slots in the conductive metal inner cylinder; providing a fourth layer within the third layer, and including a plastic tube within the sensing layer, and holding the sample in the plastic tube during measurements.

15. The method of claim 14, further comprising composing the two-port bifilar helix structure as an external feeder helix with two 3-turn helices and radiates azimuthal direction.

16. The method of claim 15, further comprising orienting the plurality of slots in the conductive metal inner cylinder as orthogonal to the rotation orientation of the two-port bifilar helix structure to enhance sensing, and the radiation from two-port bifilar helix structure including the reflected fields from the conductive metal inner cylinder leak through the plurality of slots to amplify the sensing capability and to focus the signal on the inner testing of the fourth layer.

17. The method of claim 16, further comprising supporting the cylindrical section by a base structure that is composed of 2D layers including a feeding network, a dielectric substrate, and a ground plane.

18. The method of claim 17, further comprising using the cylinder slots as complementary split resonators and detecting the concentration of urine constituents instantaneously through electromagnetic wave radiation; and urine constituents include creatinine, ammonia, urea, or uric acid; and detecting creatinine between about 0.2 mg/dl and about 4 mg/dl.

19. The method of claim 18, further comprising monitoring the creatinine level over multiple frequency bands with enhanced sensitivity with the plurality of slots in the conductive metal inner cylinder leak result in a multi-band response.

20. The method of claim 19, further comprising providing a current density surface distribution along with the electric field distribution over the inner surface of the slotted metallic cylinder that surrounds the sample shows highly concentrated areas around the slots of the inner metallic cylinder of the sensor at different frequencies, including a highly sensitive area towards changes in the constituents of the loading sample.
